(19) United States
(12) Patent Application Publication
(10) Pub. No.: US 2006/0205086 A1
(43) Pub. Date: Sep. 14, 2006

(54) Diagnostic device

(75) Inventor: Wei Hu, Toronto (CA)

Correspondence Address: BERESKIN AND PARR, 40 KING STREET WEST, BOX 401, TORONTO, ON, M5H 3Y2, CA

(73) Assignee: SPECTRAL DIAGNOSTICS INC., ONTARIO (CA)

(21) Appl. No.: 10/531,912

(22) PCT Filed: Oct. 24, 2003
(86) PCT No.: PCT/CA03/01613

§ 371 Date: May 8, 2006

Related U.S. Application Data
(60) Provisional application No. 60/497,917, filed on Aug. 27, 2003.

(30) Foreign Application Priority Data
Oct. 24, 2002 (US) 10/279,566

(51) Int. Cl. G01N 33/558

(52) U.S. Cl. 436/514; 435/287.1

(57) ABSTRACT

A diagnostic device for testing a liquid sample has a carrier for receiving at least a portion of the sample and a sample delivery means. The sample delivery means has a delivery channel that is in fluid communication with the carrier. The delivery channel has a first delivery channel surface facing a second delivery channel surface, wherein the first delivery channel surface is spaced apart from said second delivery channel surface by a distance that promotes longitudinal advancement of the sample along the delivery channel by capillary action. A deposition channel, to promote lateral dispersal of the sample across a corner, is formed either in a housing or between the housing and the carrier.

### FIELD OF THE INVENTION

This invention relates to the detection of analytes including those that are relevant in medical diagnosis. More particularly, the invention relates to devices that typically are hand-held and allow for the detection of analytes in specimens such as body fluids, environmental samples and the like.

### BACKGROUND OF THE INVENTION

A wide variety of devices for detecting the presence of analytes in a liquid sample, such as body samples and environmental samples, through the use of immunochemistry have been recently developed. Typically, for body samples, these devices perform the in vitro diagnostic test on the surface of a dry porous carrier, such as a sheet or strip of nitrocellulose membrane, contained in a housing having a sample deposition site and a detection site for viewing the assay result(s). A sample is applied as a liquid drop to one end of the carrier, and flows by capillary action downstream to the other end passing a reagent immobilized in between. As the sample advances along the strip, additional mobile reagents disposed on the carrier bind to the analyte and become entrained in the sample flow. The assay is read by observing the presence of one or more analyte-binding reagents at the detection site.

More recently, it has been observed that devices which encourage convergent sample flow through the mobile reagents on the carrier are advantageous, because such devices concentrate the reagents and analytes and retard the migration of particulates, such as red blood cells, thereby enhancing the reliability of the test results. Otherwise, the presence of red blood cells in the detection channel interferes with the proper visualization of the test results because of the intense hue of the cells.

Diagnostic devices of this type are intended to be disposable after a single use, and must therefore be designed for inexpensive production. Importantly, however, the engineering required to perform the test in a rapid and reproducible fashion, with maximum sensitivity and specificity and with minimum sample volume, is highly demanding. The art is therefore continuously refining the design of such devices in order to improve their price and practicality.

One device, which incorporates numerous improvements over those currently marketed, is described in WO00/08466 published Feb. 17, 2000 in the name of the present assignee. Described therein is a diagnostic device that, like many others, incorporates both a dry porous carrier in the form of a nitrocellulose sheet, and a housing for that carrier that incorporates both a sample inlet and a window for viewing the assay result. The sample inlet of the device is particularly unique, in providing a U-shaped channel from which sample can be deposited across a wide sample deposition zone for capillary flow into a narrowed detection channel in which the analyte is captured for detection. By channeling the flow of sample confocally through the mobile reagents located upstream in the deposition zone, the device concentrates reagents and analyte and retards red blood cell migration, and thereby enhances the sensitivity of the assay for a given volume of sample.

That device utilizes a carrier that most desirably is a uniplanar, single sheet of nitrocellulose, and uses both the housing and repellant border material to drive sample flow from the sample zone to the relatively narrow detection channel. In other devices, a multiplanar construction is incorporated in which the various pads, formed of the carrier material, are coupled in flow communication. In this arrangement, each pad can be used for a different purpose. For instance, and as shown in co-assigned U.S. Pat. No. 5,658,801, each one of a plurality of pads can be impregnated with a different one of the various reagents required to detect a given analyte by the lateral flow method. These pads can then be “stacked” one above the other and in flow communication with a base carrier. Reagents deposited in the pads are picked up by sample that has been applied to the top pad, and any complexes formed with the analyte then are captured by reagent immobilized downstream on the base carrier, where a reading can be taken.

In an alternative multi-pad design, described for instance in U.S. Pat. No. 5,559,041, a sample pad optionally impregnated with reagent is positioned in flow communication with a detection pad bearing immobilized reagent. One or more intervening pads are also incorporated, to function as a filter for particulates contained in the sample.

More recently, it has been observed that devices which encourage convergent sample flow through the mobile reagents on the carrier are advantageous, because such devices concentrate the reagents and analytes and retard the migration of particulates, such as red blood cells, thereby enhancing the reliability of the test results. The presence of red blood cells in the detection channel interferes with the proper visualization of the test results because of the intense hue of the cells.

Several devices have been developed having liquid sample deposition structures which directly or indirectly promote the convergent flow of liquid sample, or the converging of sample flow, towards the detection channel of the carrier. For instance, a device including a U-shaped sample deposition means from which sample can be deposited across a carrier for capillary flow into a narrowed detection channel produces a generally convergent sample flow. However, given that diagnostic devices of this type are intended to be disposable after a single use, and, thus, inexpensive, the engineering cost necessary to manufacture such a U-shaped sample deposition means can be prohibitively high.

Despite these advances in diagnostic devices, there remains a need to improve the ease with which they can be used and manufactured, without sacrificing their reliability. Accordingly, the object of the present invention is to provide an improved device for detection of analytes in a liquid sample. The device will encourage the efficient separation and/or filtration of particulates so as to provide reliable test results. Additionally, there remains a need for a diagnostic device having a sample delivery means which is inexpensive to manufacture and easy to use, and which facilitates the reception of liquid sample so as to provide reliable test results.

### SUMMARY OF THE INVENTION

The present invention is directed to a diagnostic device for testing a liquid sample having a carrier for receiving at least a portion of the sample and a sample delivery means.

The present device utilizes a carrier that is adapted for highly efficient sample flow, which not only maximizes use of sample volume but also maximizes analyte flow across the analyte capture line. In one embodiment of the present device, this is achieved using a carrier formed of an array of pads, including a sample pad for receiving sample, and at least one detection pad which defines a detection channel having a width that is narrower than the sample pad. In a preferred embodiment, the detection channel comprises both a detection pad and a bridging pad that is coupled in flow communication between the sample pad and the detection pad. In a preferred embodiment, the bridging pad has a lower surface that is in contact with the upper surfaces of the sample pad and the detection pad. By this arrangement, there is provided an interface that functions to filter certain particulates from the sample migrating across the carrier pad array. In a further preferred embodiment, the bridging pad and the detection pad are coupled using an upper barrier layer that is impervious to liquid. By this arrangement, capillary flow is enhanced for all sample migrating into the detection channel, thereby increasing and concentrating the flow of sample, and analyte, across the detection pad.

By providing a carrier that is modular in design and comprises individual pads, the present device can be produced with far less waste of carrier material than a single carrier sheet that performs similarly, to channel sample into a narrow detection channel. Moreover, the rate and volume of sample flow across the detection pad is increased, relative to a single sheet design, by using the overlapping pad arrangement and barrier layer by which the various pads are coupled in flow communication.

Also in the present device, there are provided certain key features that simplify manufacturing and handling of the device during use and transportation. The present device comprises a housing and a carrier material having a surface suitable for conducting the assay, which in one embodiment is constituted by the pad array just described. In the present device, the housing is provided with a sample inlet that communicates with the carrier by way of a sample deposition channel. In the present device, the sample deposition channel is adapted to deposit sample as a generally linear band having its longer axis generally transverse to the path of sample flow on the carrier. Desirably, the width of the sample band deposited from the deposition channel is greater than the width of the detection channel through which the deposited sample migrates. As distinct from sample deposition channels that are U-shaped, the present linear deposition channel offers greater ease of manufacture. Moreover, it has been found that the U-shaped design, intended to channel sample for convergent flow toward a narrow detection channel is unnecessary; sample deposited from a linear band that is perpendicular to sample flow and wider than the downstream detection channel, as in the present invention, has been found to migrate naturally toward and into the detection channel without significant loss of sample to regions of flow stagnation. Moreover, by this arrangement, reagent-bound analyte becomes concentrated at the entry to the detection channel, and thus migrates across the detection pad in concentrated form to enhance assay sensitivity.

The means by which the device housing receives sample and deposits sample onto the carrier can vary in accordance with aspects of the present invention.

Thus, in one aspect of the present invention, the sample delivery means has a delivery channel that is in fluid communication with the carrier, the delivery channel having a first delivery channel surface facing a second delivery channel surface, wherein the first delivery channel surface is spaced apart from said second delivery channel surface by a distance that promotes longitudinal advancement of the sample along the delivery channel by capillary action.

In a preferred embodiment, the distance between the first delivery channel surface and the second delivery channel surface is less than 1.0 mm, and more preferably is 0.5 mm. The delivery channel may be generally rectilinear.

The present invention is also directed to a diagnostic device for testing a liquid sample having a carrier for receiving at least a portion of the sample and a sample injection means. The sample injection means has an injection channel that is in fluid communication with the carrier. Additionally, the injection channel is in fluid communication with a sample delivery means, and wherein at least a portion of the sample received in the sample delivery means flows into the injection channel.

### BRIEF DESCRIPTION OF THE DRAWINGS

For a better understanding of the present invention and to show clearly how it may be carried into effect, reference will now be made, by way of example, to the accompanying drawings which show a preferred embodiment of the present invention in which:

FIG. 1a is an exploded perspective view of the components of a device in accordance with a first embodiment of the present invention;

FIG. 1b is a top view of the first embodiment of the device;

FIG. 1c is a top view of a base member of the housing of the first embodiment of the device;

FIG. 1d is a side view along line 1-1 of FIG. 1b;

FIG. 1e shows the sample delivery channel of the first embodiment along line 2-2 of FIG. 1b;

FIGS. 1f and 1g show perspective views of the sample delivery channel of FIG. 1e;

FIG. 2 illustrates a perspective view of a pad arrangement of the device of FIG. 1;

FIG. 3 illustrates a top plan view of the pad arrangement of the device of FIG. 1;

FIG. 4 illustrates a side elevational view of the pad arrangement of the device of FIG. 1;

FIG. 5 illustrates a perspective view of the pad arrangement including a wicking pad;

FIG. 6 is a top perspective view of the outer surface of the top member of the device of the present invention, showing the sample delivery means;

FIG. 7 is a sectional side view of the conduit of the device of FIG. 1;

FIG. 8 is a sectional side view of the device of FIG. 1 with a first alternate conduit;

FIG. 9 is a sectional side view of the device of FIG. 1 with a second alternate conduit;

FIG. 10 is a sectional side view of the device of FIG. 1 with a third alternate conduit;

FIG. 11 is a sectional front view of the injection channel apparatus of the subject device;

FIG. 12 is a sectional side view of the injection channel apparatus and sample deposition apparatus of the subject device;

FIG. 13 is a top perspective view of the inner surface of the top member of the subject device;

FIG. 14 is a sectional side view of the sample deposition apparatus of the subject device;

FIG. 15 is a top perspective view of the base member of the subject device;

FIG. 16 is a perspective, exploded view of a third embodiment of the device of the present invention;

FIG. 17 is a view of a vertical section through the device of FIG. 16;

FIG. 18 is a perspective view of the upper member of the third embodiment of the device;

FIG. 19 is a perspective view of a variant of the upper member of the third embodiment of the device;

FIG. 20 is a sectional view similar FIG. 17 showing the components separated;

FIG. 21 is a perspective view of the base of the third embodiment of the device;

FIG. 22 is a view of an enlarged scale of part of the sectional view of FIG. 17, shown inverted;

FIG. 23 shows a detail of FIG. 22 on a further enlarged scale;

FIGS. 24a and b show a perspective view and a plan view of a detail of the base member of the third embodiment;

FIG. 25 shows the detail of FIG. 23 further enlarged;

FIG. 26 shows part of FIG. 18 on an enlarged scale; and

FIGS. 27a-e show results with a device according to the first embodiment of the present invention.

### DETAILED DESCRIPTION OF THE INVENTION

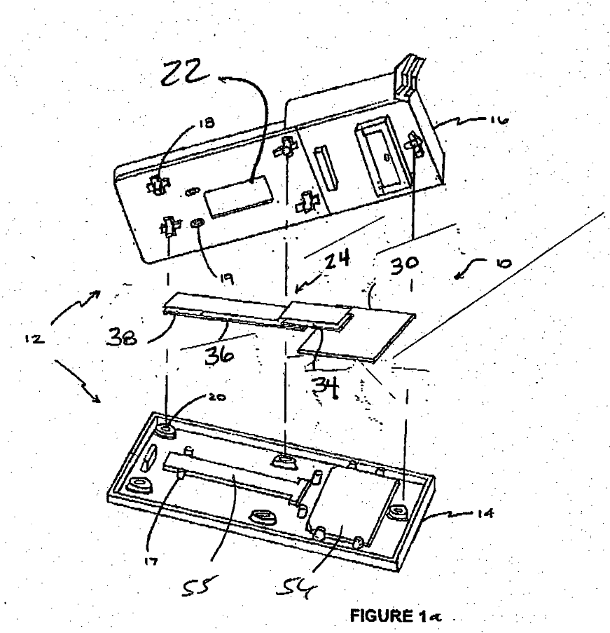

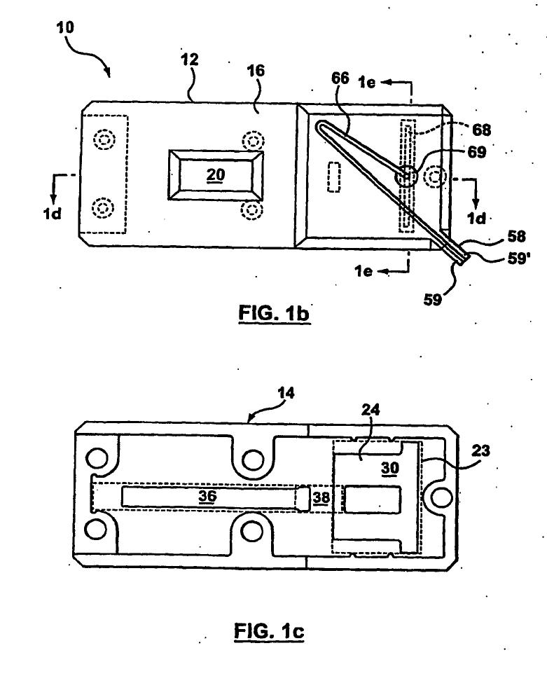

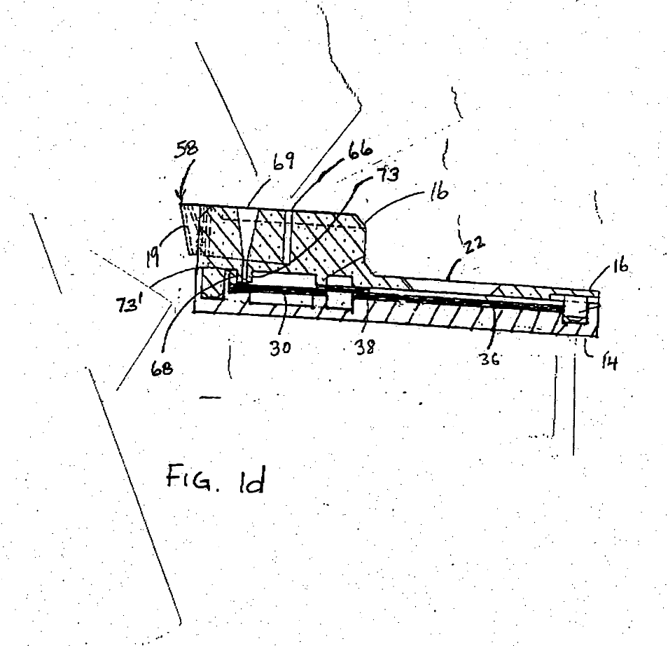

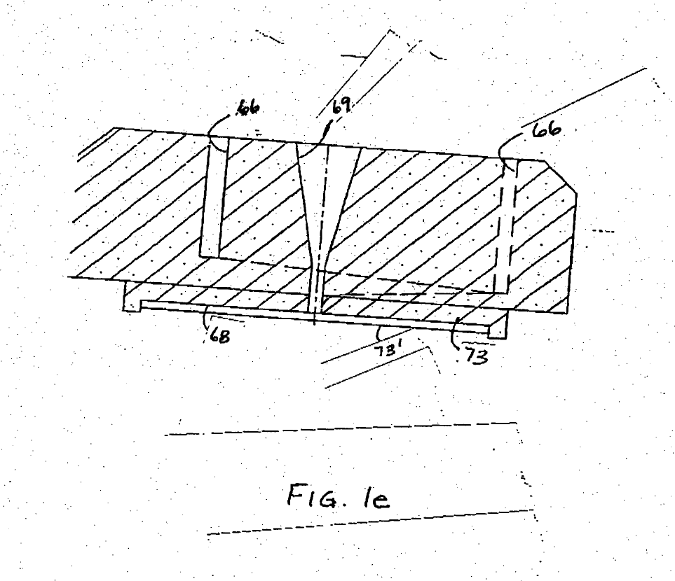

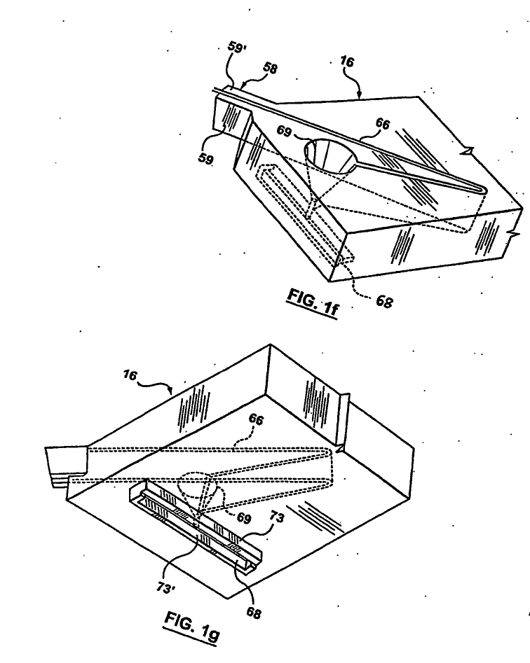

Reference is made to FIGS. 1, which shows a diagnostic device 10 made in accordance with a first embodiment of the present invention. The device 1.0 shown in FIG. 1 comprises a housing 12 formed of a base member 14 and an upper member 16 that are mateable, by friction fit, using connectors 18 provided on the upper member 16 and corresponding recesses 20 provided on the base member 14. Any other means known in the art may be employed to permanently or removably fix the members 14 and 16 together. The upper member 16 is also provided with an observation window 22 for viewing the assay results. The housing 12, and the device 10 generally, are of a size convenient for holding the device in one hand during operation of the test. A carrier 24 is mounted between the base and upper members 14, 16. Projections or carrier stabilizers 17 and corresponding alignment means or apertures 19 are provided for the carrier 24.

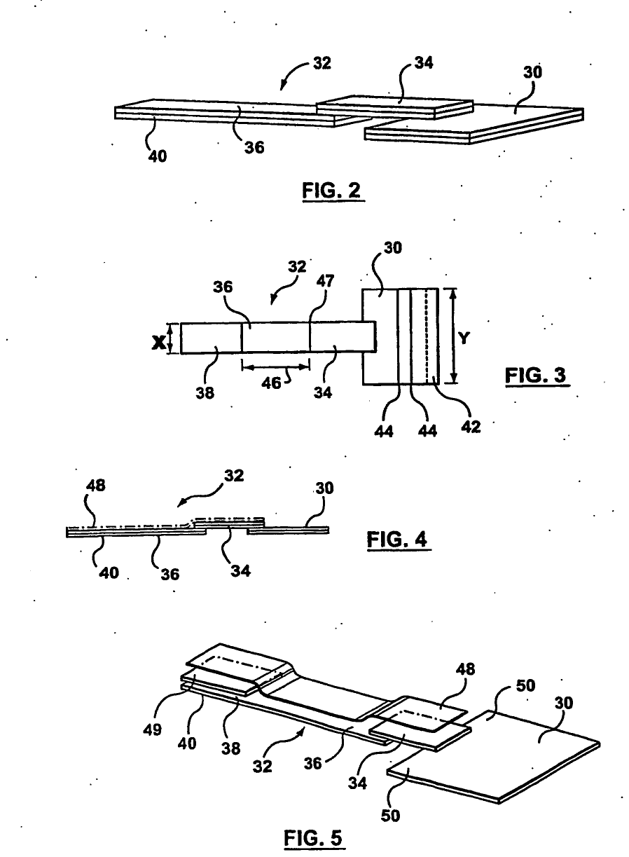

Reference is made to FIGS. 2-5 which illustrate the carrier 24 in a preferred embodiment of the present invention. Carrier 24 is comprised of an array of pads coupled in flow communication. The array comprises a sample pad 30 for receiving at least a portion of the sample and a detection channel 32. In the illustrated embodiment, the detection channel 32 is comprised of a bridging pad 34 and a detection pad 36 with a capture zone 38. Alternatively, the detection channel 32 may comprise a single detection pad having dimensions comparable to the combined detection pad 36 and bridging pad 34. To couple a single detection pad to the sample pad 30, a backing material 40 on the detection pad 36 can be removed at least at the interface thereof with the sample pad 30, to foster sample flow from the sample pad 30 into the detection pad 36 defining the detection channel 32.

The pads within the array are provided with one or more reagents 44. The reagents may be any mobile or immobile analyte-binding reagents suitable for detecting the analytes of interest and/or for eliminating interfering factors. To perform the typical lateral flow immunoassay, for instance, analyte-binding reagents, also referred to as detector reagents, are deposited on the sample pad 30 downstream from the site 42 at which sample is first applied to the carrier 24. These analyte-binding reagents are deposited, as indicated at 44, on the carrier 24 as mobile reagents that become bound to a particular analyte in the sample flow for movement with the selected analyte. These reagents also are typically coupled to a label that can be detected either visually or with suitable instrumentation. Examples of suitable analyte-binding reagents are antibodies bearing such labels as gold sol, enzyme, fluorophore, or lumiphore. Analyte present in the sample stream thus becomes bound to the labeled detector reagent to form analyte-reagent complexes. The complexes migrate into a detection zone 44, where they encounter an analyte-binding capture reagent immobilized on the carrier within view from the observation window 22 and 122. The accumulation of label at the capture zone 38 reports a positive assay result, confirming that the targeted analyte is present in the sample under investigation.

It will be appreciated that one or more different mobile detector reagents can be deposited as individual bands 46 spanning the width of the sample pad 30. Alternatively, the detector reagents can be deposited as reagent blots (not shown) on the sample pad 30.

There are numerous adaptations of this immunoassay format, including indirect formats and competitive binding formats. It will be appreciated that the particular format chosen for performing the assay is not critical to the present invention, and that any of a variety of formats can be adopted with the present device.

It will further be appreciated that the immobilized capture reagent can be deposited in the shape of any desired indicia, but is illustrated as a straight line. To control for false negative results, the detection pad 36 can further comprise a control line 47, bearing an immobilized reagent that is non-specific for analyte, but indicates that sample has migrated successfully into analyte capture zone 44. A binding partner specific for a mobile detector reagent deposited in the sample pad 30 can serve this purpose.

Each pad within the array constituting carrier 24 can be formed of the same carrier material, but this is not essential. Different carriers and carrier compositions can be used. For instance, the pads can be formed of glass fibers, of nitrocellulose, or of any suitable polymeric material on which liquid sample can flow desirably by capillary action. The carrier 24 further can be made of material suitable for filtering sample as it migrates, or for allowing sample particulates to separate chromatographically as sample migrates therealong. This is of particular benefit when the applied sample is blood. In this case, the structure of the carrier material preferably functions to separate the blood components chromatographically, causing the formation of a plasma front advancing ahead of red cells and other particulate material.

In the embodiments illustrated in FIGS. 2-5, the pads are each formed of nitrocellulose having an average pore size in the 1-10 micron range, preferably about 3-5 microns. The pads are cut from a larger sheet of nitrocellulose having backing material 40 that is water-impermeable, to prevent sample leakage, and provides some rigidity to the otherwise supple nitrocellulose material. A suitable such material is polyester film. The pads illustrated in FIG. 2 have backing material 40, and present only one surface, or face, on which the assay can be conducted. As best shown in FIG. 5, the sample conducting faces of the sample pad 30 and the detection pad 36 are bridged between and in contact with the sample conducting face of the bridging pad 34, i.e. the bridging pad 34 is inverted with its backing material 40 uppermost. The pads overlap at their edges to establish sample flow communication along the length of the carrier 24. By placing the bridging pad 34 above the sample pad 30, there is provided a capillary “lift” that assists with filtration of the sample and, in the case of a blood sample, further accentuates the chromatographic separation of the sample into a leading plasma front and trailing red blood cells.

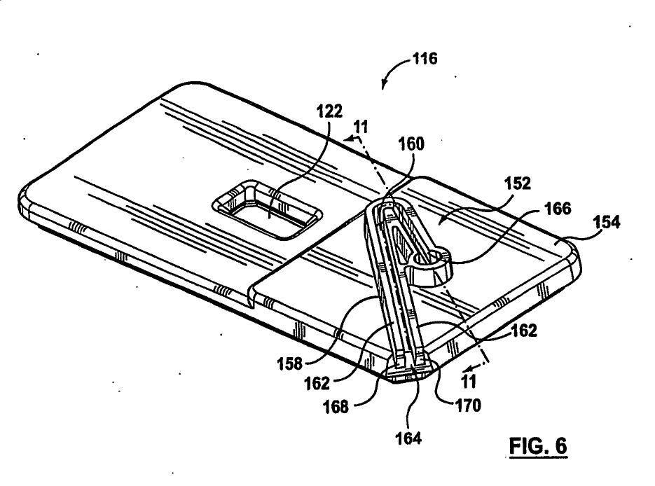

The carrier 24 has a surface area and volume sufficient to accommodate the sample volume required for a given assay. In addition, the capture zone 38 within detection pad 36 that is downstream of the detection reagents is provided with a surface area sufficient to draw sufficient sample across the capture zone 38 to detect the analyte before the carrier 24 is completely wetted by the sample, at which point, further sample migration is inhibited. This can be achieved simply by extending the length of detection pad 36, to provide for sufficient sample draw. Alternatively, and as shown in FIG. 6, the detection pad 36 can be provided at its distal end with a wicking pad 49 that is positioned in flow communication with the detection pad 36. Use of the wicking pad 49 can reduce the length of the detection pad 36, and the overall device, by substituting for an elongated detection pad.

Flow communication between each of the pads in the array can be maintained by “pinching” the pads at their overlapping edges using structure provided by the housing, as will be described in greater detail below. Alternatively, or in addition, and according to one embodiment of the present invention, flow communication between the bridging pad 34 and detection pad 36, and any wicking pad 49 present therewith, can be maintained using a layer of wettable and adhesive barrier material 48. As illustrated in FIG. 6, the layer of barrier material 48 is applied along substantially the entire length of the detection pad 36 and bridging pad 34. This ensures that these pads remain in flow communication. In addition, it has been found that the layer of barrier material 48 has the important effect of enhancing the flow of sample thereunder, and thus has the advantage of effectively drawing sample into the detection channel 32 from the sample pad 30. It will be appreciated that the layer of barrier material 48 should be either translucent or transparent, so that it does not mask the assay result from being viewed through observation window 22 and 122 (see below). Any transparent or translucent barrier material that functions like adhesive tape, such as Scotch® tape, can be used for this purpose. This barrier material 48 also prevents accidental smearing or damage or other exposure to the carrier 24 exposed at the observation window 22 and 122.

The sample pad 30 can have a width that is greater than the detection channel 32 coupled to it. As shown in FIG. 4, the detection channel 32 has a transverse width X that is reduced relative to the width Y of the sample pad 30. This is an important feature of the present carrier system. By this design, sample deposited across the sample pad 30 has been found to migrate in the direction of the detection channel 32, with minimal, if any, sample stagnation occurring in each “shoulder” 50 of the sample pad 30. In fact, any slowing of sample flow at these “shoulders” 50 provides the benefit that the sample becomes enriched for analyte/label complexes which then can flow in concentrated form into the detection channel 32, thus enhancing assay sensitivity.

Reference is made to FIG. 2 which illustrates a diagnostic device 110 made in accordance with a second embodiment of the present invention. The device 110 comprises a housing 112 formed of a based member 114 and an upper member 116 that are mateable, by friction fit, using connections 118 provided on the upper 116 and corresponding recesses 120 provided on the base member 114. The upper member 116 is also provided with an observation window 122 for viewing the assay results. The housing 112, and the device 110, are also of a size convenient for holding the device in one hand during operation of the test. As will be described in greater detail, the base members 14 and 114 the upper members 16 and 116 have been formed with different structural elements to facilitate the reception and deposition of liquid sample so as to provide reliable test results.

The housing 12 and 1 12 accommodate a porous carrier 24 which is held between base member 14 and 114 and upper member 16 and 116. The carrier 24 has a surface for conducting sample flow between the sample deposition site and the analyte detection site situated within the observation window 22 and 122. The carrier 24 is maintained in the housing 12 and 112 in sufficient alignment to allow the diagnostic test to be performed using a plurality of carrier stabilizers 17 and 126 formed on the base member 14 and 114. At least one alignment means 19 and 128, each corresponding to one carrier stabilizer 17 and 126 is provided on the top or upper member 16 and 116.

This second embodiment for depositing sample onto the carrier 24 is now described in greater detail.

As shown in FIG. 1, the carrier 24, bearing mobile detection reagents on the sample pad and immobilized capture reagents on the detection pad, is received between base member 14 and upper member 16, so that the capture reagent line and any control reagent line are positioned for viewing at window 22. The carrier 24 is registered within the housing by projections 17 formed in base member 14, which abut the periphery of the carrier, to avoid continuous lines of contact with the housing. [FIG. 4]. Also provided in base member 14 are raised platforms or stages 54 and 55, which support the sample pad 30 and detection pad 36, respectively. Also provided directly under the bridging pad at its interfaces with the sample pad and the detection pad are supporting webs.

As shown in FIG. 1, upper member 16 of the present device has a sample receiving port 58 that communicates with the carrier 24 via a sample deposition means, which comprises a conduit 66 formed within housing upper member 16. Conduit 66 feeds into sample reservoir 69 which communicates with and feeds into a sample deposition channel 68, from which sample is ultimately deposited onto carrier 24.

The first embodiment of the sample delivery system is shown in greater detail in FIGS. 1d-1g. In the illustrated design, delivery of sample onto the carrier is achieved using a system that drives sample flow by exploiting a combination of surface tension minimization, capillary action, and gravity. More particularly, the conduit 66 extends externally to the sample receiving port 58 which is defined by first and second flanges, 59 and 59′. One of the two flanges can be notched to maximize port surface area and thus encourage liquid sample to minimize surface tension by moving into conduit 66. Conduit 66 is formed as a trench within the upper member 16, having a bottom, parallel side walls and a top that is open to the air. The bottom of conduit 66 is shaped to provide increasing depth along the length thereof (revealed in FIG. 1e). Thus, when the device is placed on a horizontal surface, there is a gravitational tendency for sample to flow away from the sample receiving port and into the sample deposition channel. In addition, conduit 66 is reticulated, so that when device 10 is held by the user in a vertical orientation, as would be desirable when applying a sample to be tested, there is a further tendency for sample to move by gravity toward the sample reservoir 69. This is achieved by forming the conduit 66 as a trough that is descending, when the device is held in the vertical position. The conduit 66 desirably has a volume sufficient to hold sample sufficient for performance of a given test. It will be appreciated that this volume can be adjusted by increasing the length of the conduit. As shown in the Figures, this is achieved by reticulating the conduit, in the shape of a hair-pin as shown. Any other design, linear or curved, could be adopted to this end. In addition, it will be appreciated that the upper member 16 can include a protective layer of material, such as a fixed or removable adhesive tape (not shown), to cover the exposed conduit and reservoir and prevent contamination. The protective layer is desirably translucent, so that accumulation of sample in the reservoir can be viewed by the user.

The conduit 66 terminates at and feeds into sample reservoir 69. The open end of conduit 66 is formed within the side wall of the reservoir 69. As shown in FIGS. 1d and 1e, reservoir 69 is conically shaped, i.e., has a surface area that is increased relative to conduit 66, but thereafter decreases toward the sample deposition channel. At its top, reservoir 69 is open to the air. At its bottom, reservoir 69 opens into a channel extending laterally therefrom, which forms the linear sample deposition channel 68. As shown in FIGS. 1d and 1e, the leading side wall 73 of the deposition channel is resected, so that carrier positioned thereunder abuts the longer trailing side wall 73 and thereby is registered directly under the opening of the channel, to receive sample therefrom. This beveling of the side walls forming the sample deposition channel further ensures that sample deposited from the channel is free to flow forward along carrier 24, and so that flow is resisted in the opposite direction.

By this design, sample received at port 58 is drawn by surface tension minimization, capillary action and gravity into conduit 66, flows therealong by capillary action and gravity into reservoir 69 which then fills and empties by gravity and capillary action into channel 68 which then fills by capillary action until sample meets carrier 24 and is drawn thereonto for movement by capillary flow into the detection channel and across the capture reagent line.

The performance of a typical analyte detection test involves depositing at least a portion of a liquid sample on sample pad 30 having the one or more mobile reagents provided thereon. These reagents bind to analytes present in the liquid sample to form analyte-reagent complexes and becomes entrained in the sample flow for movement with the analytes. The complexes advance with the liquid sample flow by capillary action towards bridging pad 34 and migrate into the detection channel 32. In the detection channel 32, the complexes encounter one or more analyte-binding capture reagents immobilized on capture zone 38. The accumulation of each label from the analyte-reagent complexes at a respective immobilized capture reagent in the capture zone 38 reports a test result, confirming that either the targeted analyte or one of the mobile reagents is present in the liquid sample under investigation. An observation window 22 and 122 may be provided in top member 16 and 116 corresponding to capture zone 38 to allow the results of the test to be viewed either visually or with suitable instrumentation. The detection of the presence of at least one analyte enables a physician to characterize the cardiac event, for example, as stable or unstable angina or as a myocardial infarction.

The reliability of a diagnostic test of this type depends on depositing a sufficient amount of liquid sample on the carrier, separating any particulates, such as red blood cells, from the liquid sample so as to permit the plasma containing the labeled analyte-reagent complex to advance to the detection channel, and blocking interfering factors. Efficient separation of red blood cells from the liquid sample is particularly important because red blood cells are strongly coloured and, thus, tend to interfere with the viewing and interpretation of the test results.

In embodiments of the device 10, 110 of the present invention provides improved structures for the delivery and deposition of liquid samples on carrier 24, and particulate filtration and separation structures that more efficiently remove particulates from the liquid sample, such structures are now being described in greater detail.

Reference is now made to FIGS. 6 to 15, which illustrates a sample delivery means 52 made in accordance with a second embodiment of the present invention. FIG. 6 shows the top member 116 of a device 110 having an outer surface 154 and an inner surface 156. The outer surface 154 is provided with the sample delivery means 152. The sample delivery means 152 comprises a conduit 158 with a generally V-shaped (in plan view) channel having a bed 160, a pair of side walls 162, and a top that is open to the ambient environment. The bed 160 of conduit 158 is designed to provide increasing depth along the length thereof. As best shown in FIG. 11, bed 160 ha a depth that increases at an angled slope 149 along the length of the conduit 158. Accordingly, when the device is placed on a horizontal surface, there is a gravitational tendency for sample to advance along the conduit, aided by capillary action.

Returning to FIG. 6, the conduit 158 further comprises an inlet 164 and an outlet 166. The inlet 164 extends outwardly from outer surface 154 and comprises flanges 168 and 170. The inlet 164 receives the liquid sample that is to be tested. At least a portion of the sample received at the inlet 164 advances along the conduit 158 to the outlet 166. Flanges 168 and 170 are notched to facilitate liquid sample entering into conduit 158.

Additionally, the conduit 158 has a generally V or an open hairpin shape such that when the device 110 is held by the user in an angled orientation, as may be desirable when a liquid sample is received in the conduit for testing, there is a further tendency for at least a portion of the sample to move by gravity along the conduit. Any other suitable conduit design may be adopted to this end, including, for example, a linear or curved conduit.

To encourage this type of flow, it will be appreciated that top members 16 and 116 may be suitably formed of any material that is wettable and can be machined or otherwise shaped to introduce the features of the present sample delivery system. Suitable such materials are in common use in the diagnostics industry and include hydrophilic plastics material, such as acrylic, including methacrylates and polymethacrylates. Conversely, the base members 14 and 114 of the housings 112 and 112 may be desirably formed of machinable, hydrophobic plastics material to repel diffusion of sample onto the base member from the carrier 24. Suitable such materials include polystyrene.

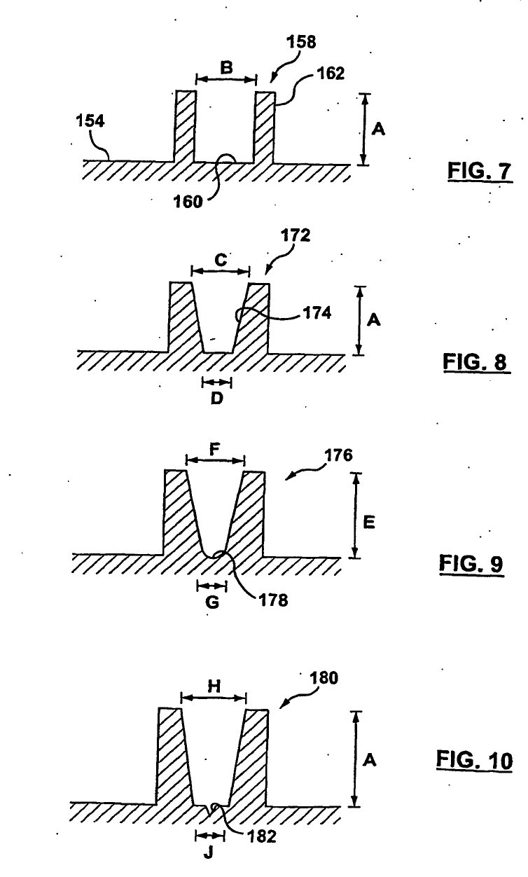

FIG. 7 shows one profile for the conduit 158, comprising the bed 160 and the side walls 162, or flanges 168, 170 having a deep rectangular cross-section. Conduit width B should preferably be less than the conduit depth A so that the ratio of B to A is equal to or less than 1.0. Most preferably the ratio of B/A is equal to or less than 0.5. The conduit 158 desirably has a volume capable of holding sufficient liquid sample for the performance of any given diagnostic test. It will be appreciated that this volume can be adjusted by increasing one or more of the width, depth or length of the conduit. It should be appreciated that the conduit 158 can have various cross-sections. FIGS. 8, 9 and 10 illustrate a first, second, and third alternate conduit. Referring to FIG. 8, a first alternate conduit 172 has a trough-like cross-section with a top width C and a bottom width D, wherein the top width C is greater than the bottom width D. The first alternate conduit 172 has outwardly bowed or slanted side portions 174. FIG. 9 shows a second alternate conduit 176 comprising a depth E, top width F and a bottom width G. The top width F is greater than the bottom width G. The second alternate conduit 176 has a curved or semi-circular base 178 which reduces the amount of sample that may stagnate in the conduit. FIG. 10 illustrates a third alternate conduit 180 having a trough-like cross-section and a top width H and bottom width J. Bottom width J is less than top width H, and bottom width J is provided with a V-shaped groove along the base 182 of the conduit. A conduit shaped in accordance with the third alternate conduit 180 is also efficient at minimizing the amount of stagnation that occurs in the conduit.

Additionally, it will be appreciated that the top member 116 may include a protective layer of material, such as a fixed or removable plastic shield or adhesive tape (not shown), to cover the exposed conduit 158 to prevent contamination and tampering of the liquid sample. The protective layer is preferably translucent to enable the viewing of the sampling in the conduit by the user.

The outlet 166 of the conduit 158 is in fluid communication with an injection channel means 184. The injection channel means 184 of the device 116 extends from the outer surface 154 through to the inner surface 156 of the top member 116. The injection channel means 184 comprises a reservoir 186, a capillary injection channel 188 and an injection aperture 190. The reservoir 186 is conical or funnel shaped having a circumference that decreases toward the injection channel. Flow of at least a portion of the sample through the reservoir 186 to injection channel 188 is achieved by exploiting a combination of surface tension minimization, capillary action, and gravity.

The reservoir 186 may be designed to have a volumetric capacity that is capable of controlling the flow of the liquid sample into the injection channel 188 while, together with conduit 158, containing sufficient liquid sample for performing the diagnostic test. In general, modulation or control of the flow of the liquid sample may be achieved by different means. For instance, the flow may be controlled by constricting the cross-section of the injection channel through which the liquid sample flows.

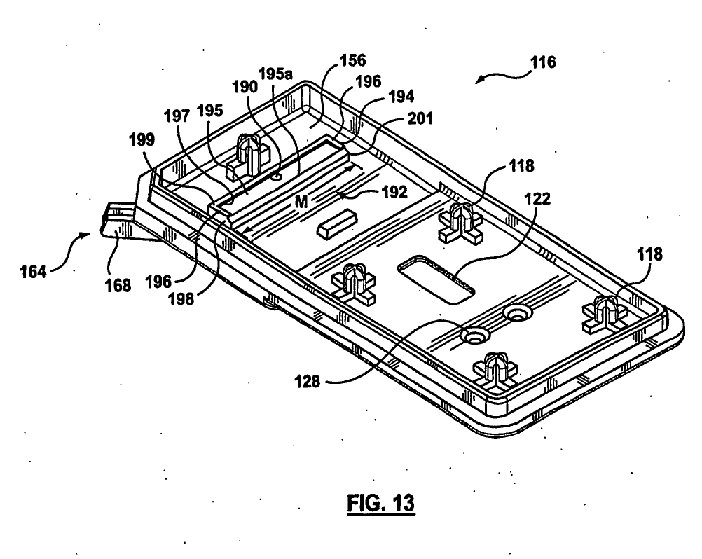

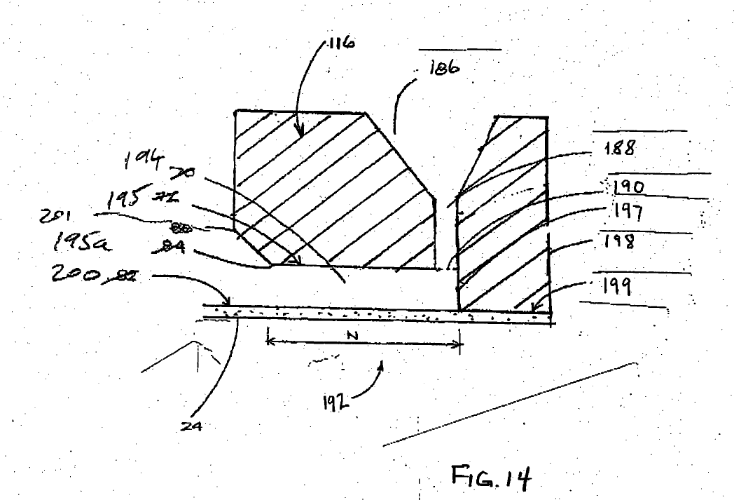

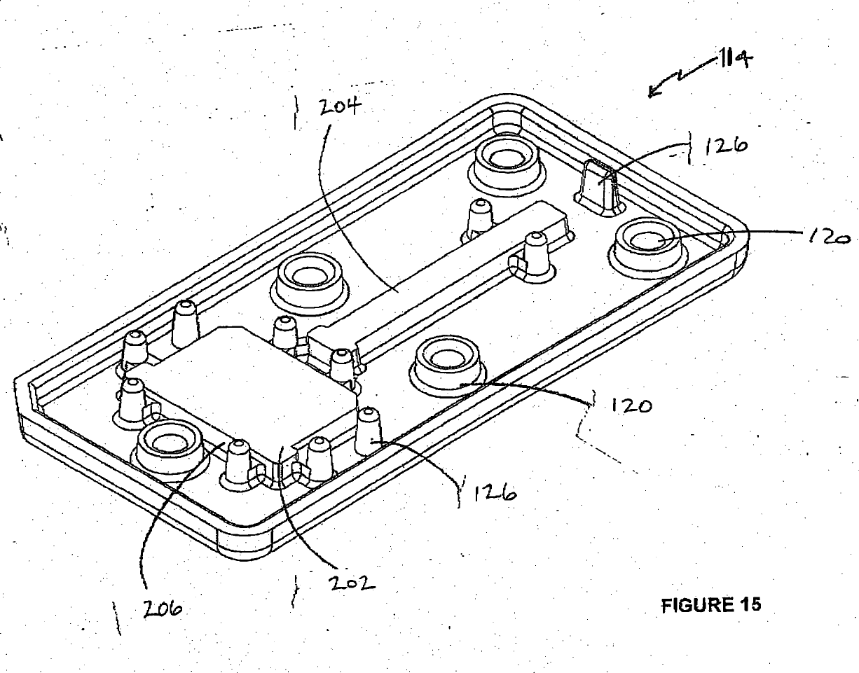

As shown in FIGS. 13 and 14, the injection aperture 190 of injection channel 184 is in fluid communication with a sample deposition means 192 provided in the inner surface 156 of top member 116. The injection aperture 190 is shown centered in the sample deposition means 192. However, it will be appreciated that injection channel means 184 may be designed to provide a sample flow to the sample deposition means 192 at any position. By the design illustrated, the liquid sample received at inlet 164 is drawn by surface tension minimization, capillary action and gravity into the conduit 158, advances therealong by capillary action and gravity through the outlet 166 into reservoir 186. The reservoir 186 then fills and empties by surface tension minimization, gravity and capillary action into the injection channel 188, and then passes through the injection aperture 190 onto the sample deposition means 192, which then fills by capillary action between the sample deposition means 192 and sample pad 24.

FIGS. 13 and 14 further illustrate that the sample deposition means 192 formed on the inner surface 156 of top member 116 has a deposition channel 194 and a deposition channel defining surface 195 which extends over at least a portion of sample pad 24 of carrier 22. The deposition channel 194 is closed at both ends 196 and has an inner wall 197, an outer wall 198 and a sill 199. The sample pad 24 comprises an operative surface 200 which is positioned to contact sill 199 of sample deposition means 192 so that sample pad 24 is registered directly under injection aperture 190, to receive sample therefrom. The shape of deposition channel 194 corresponds to the shape of the deposition channel defining surface 195. The deposition channel defining surface 195 has a beveled, trailing edge 195a.

Abutment of the sill 199 and the operative surface 200 of sample pad 24 defines the depth of the deposition channel 194, such that deposition channel defining surface 195 is spaced apart from operative surface 200 allowing at least a portion of the liquid sample flow from injection aperture 190 to disperse in deposition channel 194. Preferably, the deposition channel 194 between the deposition channel defining surface 195 and the sample pad 24 of carrier 22 has a depth equal to or less than 1.0 mm. More preferably, the depth between the deposition channel defining surface 195 and sample pad 24 is equal to or less than 0.5 mm, and most preferably the depth is 0.1 mm. The flow of liquid sample is confined to the deposition channel 194 due to the effect of capillary forces, such as a capillary trap, which delay the liquid sample from advancing downstream towards the detection channel 32 beyond the trailing edge 195a of the deposition channel defining surface 195, at least until the deposition channel is substantially filled with sample.

As shown in FIGS. 13 and 14, the width M to length N of the deposition channel defining surface is preferably 10. More preferred, the ratio M/N is equal to or less than 7. As such, the walls 197 and 198 and trailing edge 195a end at an air junction edge 201, which also delays the liquid sample from flowing downstream beyond the walls and deposition channel defining surface 195, thereby allowing the liquid sample to continue to disperse by capillary action in the deposition channel 194. The liquid sample continues to disperse in and fill the deposition channel 194 to form a generally linear sample band. The sample band corresponds to the shape of the deposition channel defining surface 195 of the deposition channel 194.

This second embodiment of the deposition channel avoids the need to machine a capillary channel that is integral within the top member itself, so tha sample is distributed uniformly within the channel before being permitted passage onto the carrier. Rather, it is now realized that it is simpler to form a capillary channel, to promote lateral flow of the sample, between the channel defining surface 195 and the pad 24. The capillary action causing lateral distribution of the sample is rapid enough that the sample starts to be absorbed into the pad 24 across the entire width of the pad 24 almost simultaneously, i.e. the portion of the sample first contacting the pad 24 adjacent the injection channel 188 does not have any significant lead in absorbing into the pad 24.

In operation, at least a portion of the liquid sample enters the sample deposition means 192 through injection aperture 190 and rapidly disperses by capillary action to the ends 196 of the deposition channel 194. Once the deposition channel 194 has been substantially filled and, accordingly, the capillary draw of the sample pad 24 exceeds that of the deposition channel 194, at least a portion of the sample band advances downstream towards detection channel. The advancing sample band initially advances from deposition channel 194 as a generally linear band which may have a slightly curved liquid frontier with the leading edge at or near the center.

The purpose of the sample deposition means 192 is now apparent. If, in the absence of the capillary trap formed by the deposition channel 194, as defined by the sample pad 24 and the sample deposition channel defining surface 195, an operative surface 200 of sample pad 24 was in direct contact with the injection aperture 190, at least a portion of liquid sample in sample delivery means would flow immediately downstream towards detection channel 32. This uncontrolled liquid sample flow would result in a greater amount of red blood cells advancing to the capture zone, since a narrow width of the flow of liquid sample would not allow the efficient separation of particulates from the plasma front by operative surfaces of the carriers. Additionally, a liquid sample flow having a narrow width may increase the duration of the diagnostic test, and reduce the concentration of analyte crossing the test line.

FIG. 15 shows the configuration of the top surface of the base member 114. A flat substantially rectangular area 202 and an elongated area 204 projecting from the top surface of base member hold the porous carrier 24. The height of the sides 206 of the rectangular area 202 is sufficient such that the operative surface 200 of the porous carrier 24 is at the same level as sill 199 of the deposition channel 194. Other configurations are contemplated by the present invention, such as designing the base and top members to hold carrier 24 in the correct orientation such that the recessed rectangular area 202 and elongated area 204 are not necessary. It is only necessary that the base and top members correspond with carrier 24 in between to define the fluid path from injection channel 188 to detection channel 32. The thickness of the various channels and areas 202 and 204 may be adjusted accordingly.

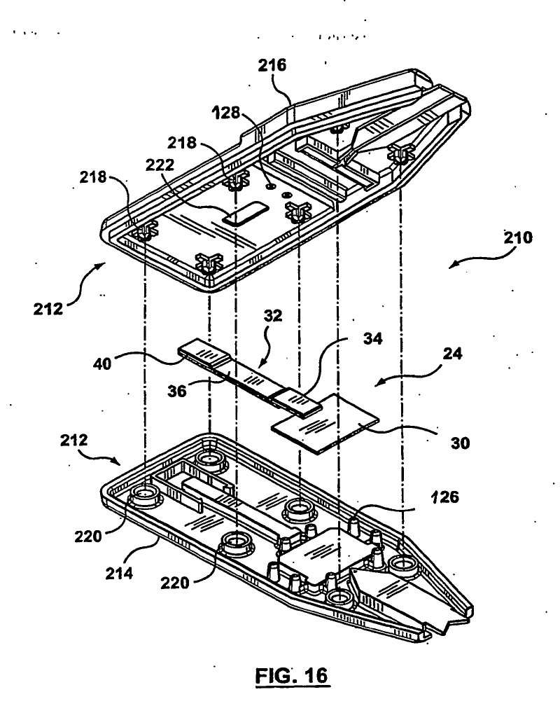

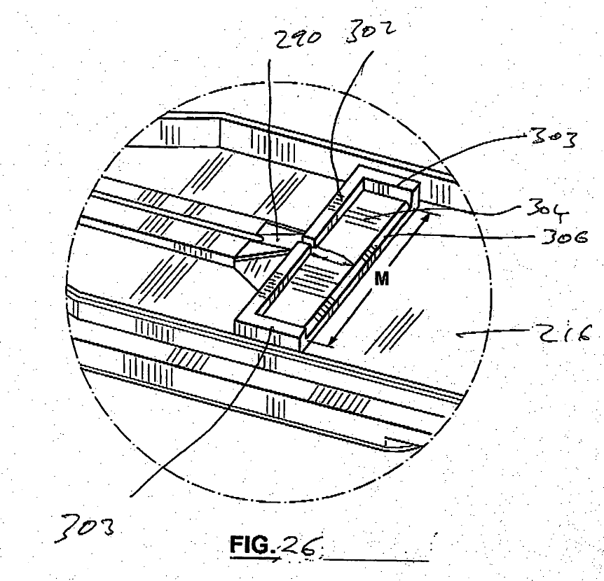

Reference is made to FIGS. 16 to 26 which illustrate a third embodiment of the device, here indicated at 210, in accordance with the present invention. The base member 214 and the upper member 216 of the device 210 have a sample delivery means 252 provided therein. The sample delivery means 252 comprises a delivery channel 254 having a first delivery channel surface 256 and a second delivery channel surface 258. The first delivery channel surface 256 is provided on an inner surface 260 of the upper member 216. The second delivery channel surface 258 is provided on an interior surface 262 of the base member 214. The delivery channel 254 is formed by mating the base member 214 with the upper member 216 using the connectors 218 and recesses 220 so that the second delivery channel surface 258 is registered under the first delivery channel surface 256. The shape of the delivery channel 254 corresponds to the shape of the first and second delivery channel surfaces 256 and 258. Preferably the shape of the delivery channel 254 is generally rectilinear.

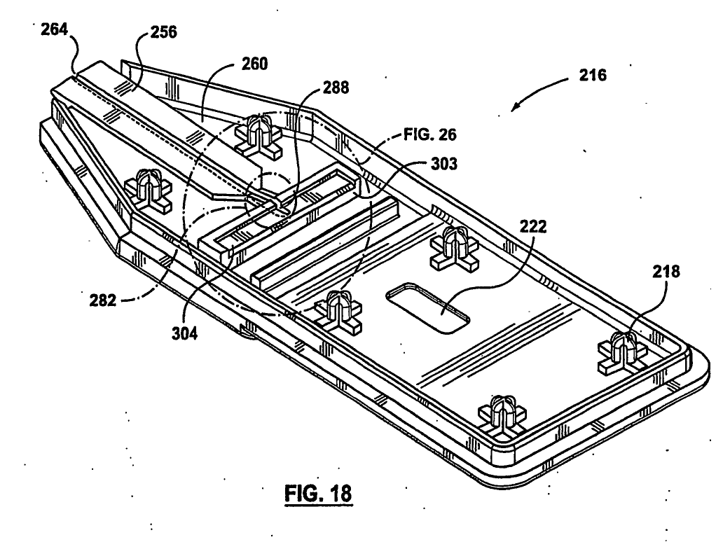

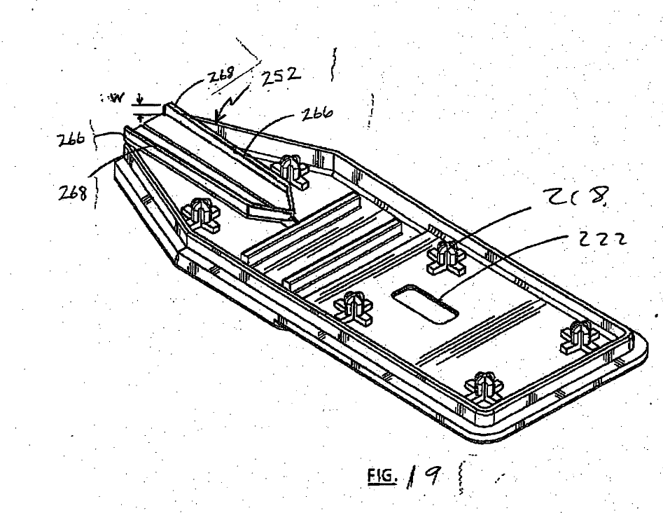

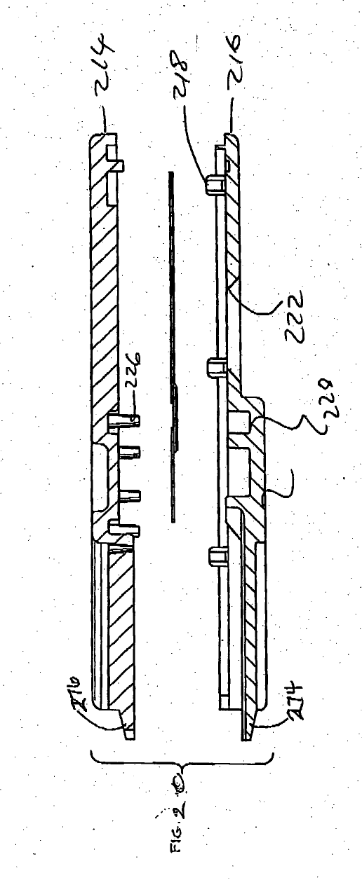

Referring to FIG. 18, an advancement groove 264 can be provided along the first delivery channel surface 256 to minimize the amount of stagnation that may occur in the delivery channel 254. FIGS. 18 and 19 show one profile of the first delivery channel surface 256 having a V-shaped advancement groove 264. It will be appreciated that the advancement groove 264 may have any suitable shape.

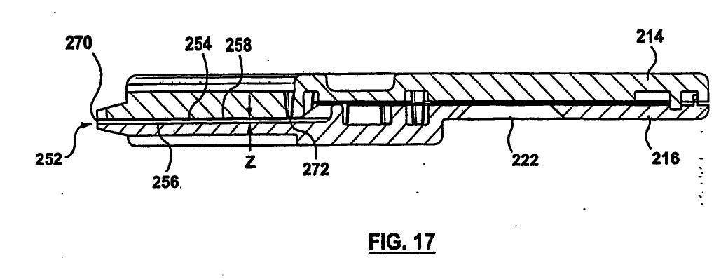

As best shown in FIG. 17, the first delivery channel surface 254 is spaced apart from the second delivery channel surface 256 by a distance Z that is designed to promote the longitudinal advancement of the sample along the delivery channel 254 by capillary action. Preferably, the distance Z between the first and second delivery channel surfaces 256 and 258 is equal to or less than 1.0 mm. More preferably, the distance between z in the surfaces 256 and 258 is equal to or less than 0.5 mm. The detection channel 254 desirably has a volume capable of holding sufficient liquid sample for the performance of any given diagnostic test. It will be appreciated that this volume can be adjusted by increasing or decreasing the dimensions of the first and second delivery channel surfaces 256 and 258.

In an alternate embodiment illustrated in FIG. 19, the sample delivery means 252 further comprises a pair of side walls 266 which define the capillary area of the delivery channel 254. The side walls 266 may have operative edges 268 which contact the first delivery channel surface 256 and/or the second delivery channel surface 258. The side walls 266 may also have a height W that corresponds with the desired distance Z between the first and second delivery channel surfaces 256 and 258 to enhance the capillary action within the delivery channel 254, and to ensure that the desired distance Z is accurately maintained.

To encourage this type of flow, it will be appreciated that upper and base members 14, 114, 214,16, 116 and 216 may be formed of any material that is wettable and can be machined or otherwise shaped to introduce the features Of the present sample delivery means 52, 152 and 252. Suitable materials are in common use in the diagnostics industry and include hydrophilic plastics material such as acrylic, including methacrylates and polymethacrylates. Conversely, the upper and base members 14, 114, 214, 16, 116 and 216 may be formed of machinable, hydrophobic plastics material, to reduce any tendency for the sample to diffuse from the carrier onto the members. Suitable such materials include polystyrene.

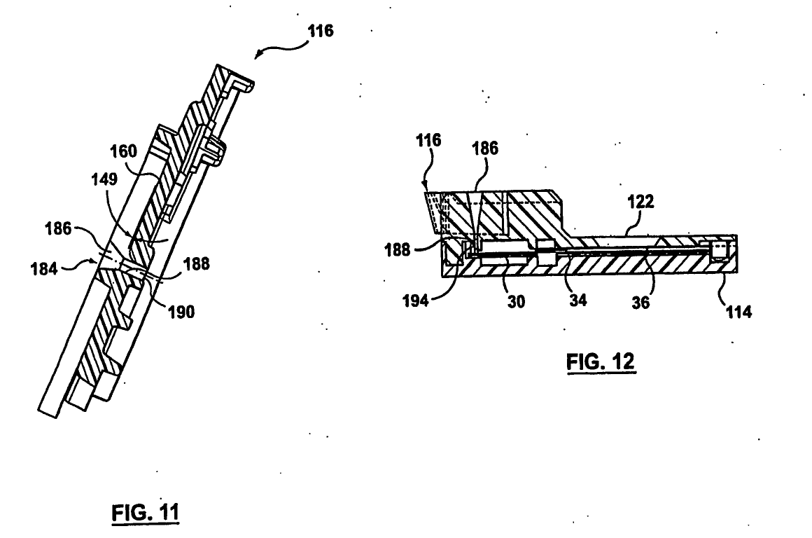

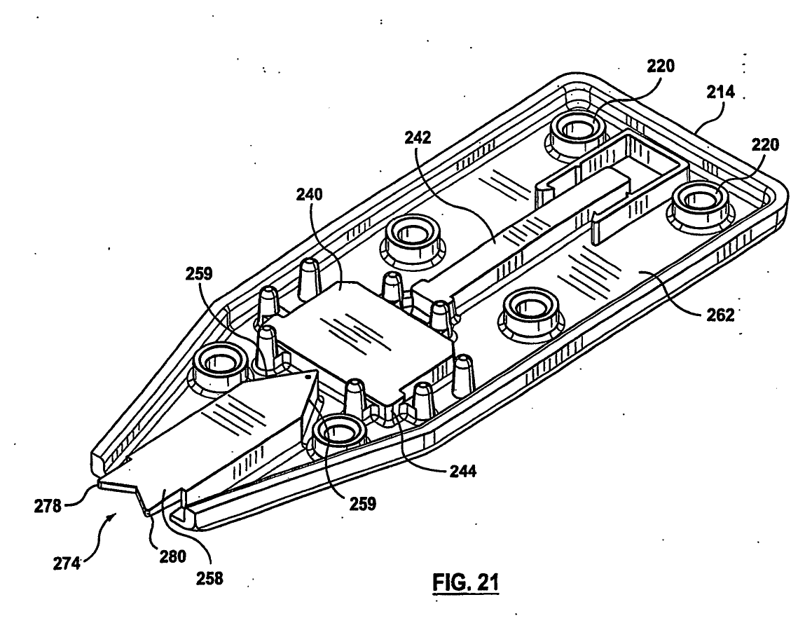

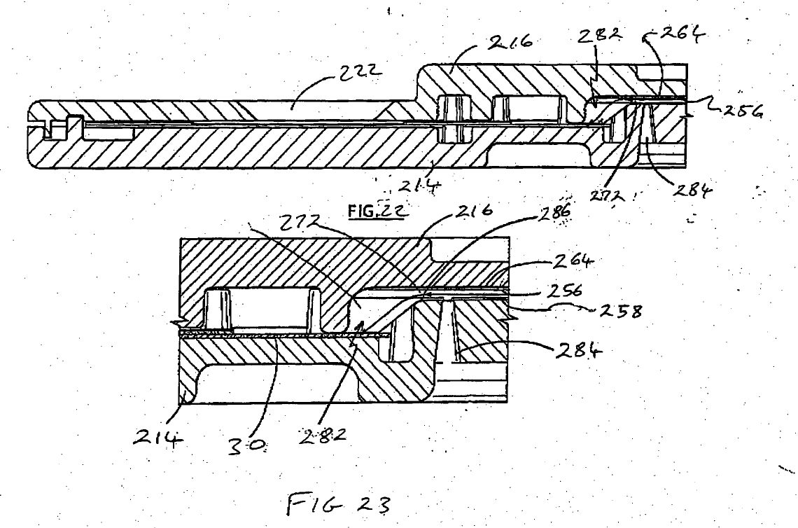

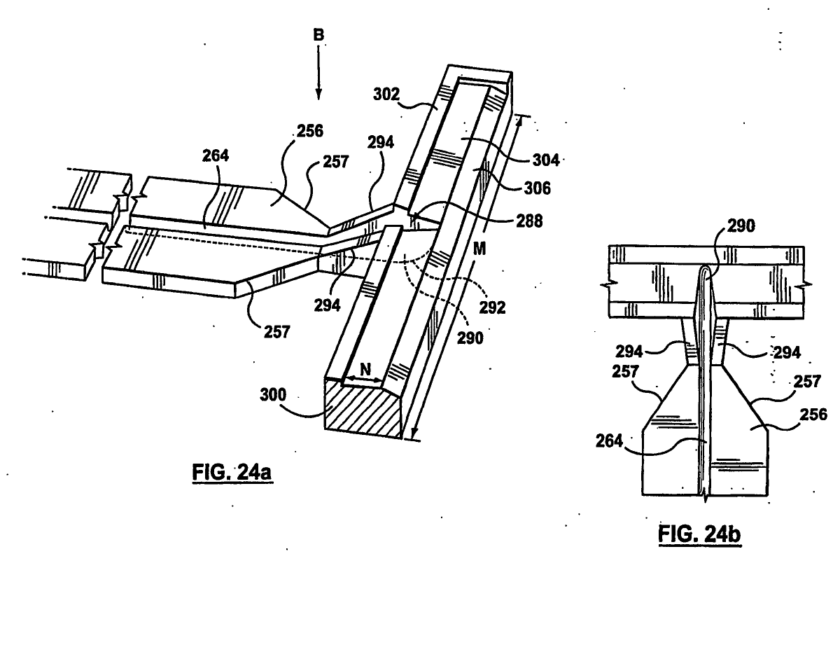

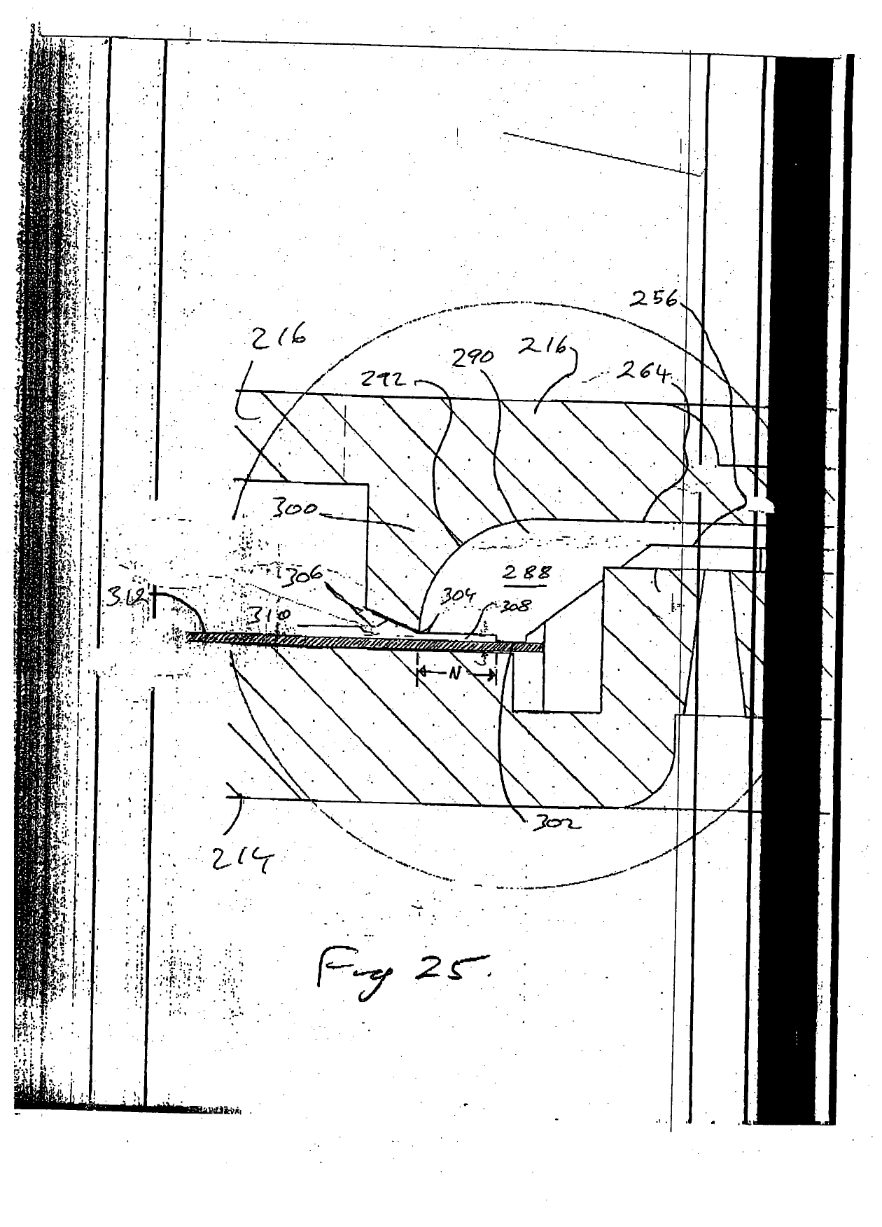

Referring to FIGS. 12, 21, 22, 23, 24, 25 and 26, the delivery channel 254 further comprises an inlet 270 and an outlet 272. The inlet 270 receives the liquid sample that is to be tested. As best shown in FIG. 21, the inlet 270 comprises an upper inlet 274 and a base inlet 276. The upper and base inlets 274 and 276 extend outwardly from the upper and base members 216 and 214, respectively, of the device 210. As shown in FIGS. 23 and 24, the upper inlet 274 includes a pair of inclined end surfaces 278 and 280 that form a notch to facilitate the receipt of liquid sample into the delivery channel 254 through capillary action and surface tension minimization. At least a portion of the sample received at the inlet 270 advances between the first delivery channel surface 256 and the second delivery channel surface 258 toward the outlet 272. The flow of at least a portion of the sample received at the inlet 270 along the delivery channel 254 is achieved by exploiting the capillary action formed by the first and second delivery channel surfaces 256 and 258.

In use, the device 210 is first turned largely upside down, so that the notch, formed at 278, 280, in the base member 214 is viewable by the user. This notch is then, typically, pressed against a pierced skin surface, to obtain a blood sample. At least a portion of the sample contained in the delivery channel 254 advances beyond the outlet 272 in the advancement groove 264. Once the liquid sample has substantially filled the delivery channel 254 and at least a portion of the advancement groove 264, as may be determined using a volume indicator 284, the device 210 is inverted, to an upright position. The act of inverting the device 210 causes at least a portion of the liquid sample contained in the advancement groove 264 beyond the outlet 272 to flow by gravity into an injection channel means 282. The remaining sample in the delivery channel 154 then empties by gravity and surface tension minimization into an injection channel means.

Referring to FIGS. 22 to 26, the outlet 272 of the delivery channel 254 is in fluid communication with an injection channel means 282. The outlet 272 has a flow edge 284 at the junction of the delivery channel 254 and the injection channel means 282 which delays the liquid sample from flowing downstream beyond the outlet 272. The second delivery channel surface 258 may be provided with a volume indicator 284 proximate to the outlet 272 to enable a user to determine whether the flow of sample has advanced to the outlet 272.

Reference is made in particular to FIGS. 22 to 26 which illustrate the injection channel means 282made in accordance with the third embodiment of the present invention. The injection channel means 282 comprises an injection channel 288, formed in an injection groove portion 290, that has tapered or inclined side walls that are an extension of the advancement groove 264. The bottom of the advancement groove 264 is rounded, as is the bottom of the injection groove portion 290 (FIG. 24a is shown in partial section to show this feature). The injection groove portion 290 ends in a curved end surface 292. Side walls 294 of the injection groove portion 290 incline downwardly (with reference to the ordinary orientation as in FIG. 22), so that the depth thereof increases in a downstream direction.

Each of the first and second delivery channel surfaces 256, 258 has respective tapered end edges 257, 259, to cause a capillary flow of liquid to be focused or guided towards the outlet 272 and into the injection groove portion 290.

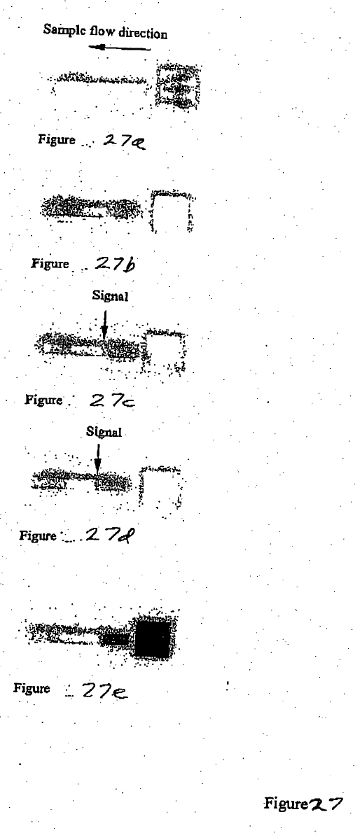

A transverse element 300 of the upper member 216 has a downwardly facing side (again as viewed in FIGS. 22, 23 25; FIGS. 24a, b showing member 216 inverted), that includes a horizontal sill 302, a deposition channel defining surface 304 and a beveled trailing surface 306. As shown in FIG. 26, the sill 302 can include end walls 303, to control flow of the sample.

The sill abuts the end of the sample pad 30, so as to define the depth of a capillary deposition channel 308. As shown in FIG. 24, the injection channel 288 of the injection groove portion 290 opens across the full width of the capillary deposition channel 308 (and also extends through the sill 302 but is closed off by the sample pad 30). From the end of the deposition channel 308, a tapered opening 310 is formed between the beveled trailing surface 306 and the sample pad 30.

Flow of at least a portion of the sample from the delivery channel 254 through the injection channel 288 of the groove portion 290 to the capillary deposition channel 308 is achieved by exploiting a combination of surface tension minimization, capillary action and gravity.

The capillary deposition channel 308, may be designed to have a volumetric capacity that is capable of controlling the flow of the liquid sample onto the carrier 24 while, together with delivery channel 254, containing sufficient liquid sample for performing the diagnostic test. In general, modulation or control of the flow of the liquid sample may be achieved by different means. For instance, the flow may be controlled by constricting the cross-section of the injection channel 288 through which the liquid sample flows.

The injection groove portion 290 is shown centered in the transverse element 300. Once again, it will be appreciated that injection groove portion 290 may be designed to provide a sample flow to the capillary deposition channel 308, at any position.

In this third embodiment, with the device 210 initially inverted, the liquid sample received at inlet 270 is drawn by surface tension minimization and capillary action into the delivery channel 254, advances therealong by capillary action to the outlet 272 and into at least a portion of the advancement groove 264 extending beyond the outlet 272. The device 210 is then inverted again, back to an upright position, causing the sample flow from the delivery channel 254 into the injection channel 288 of the injection groove portion 290. The injection groove portion 290 then empties by surface tension minimization, gravity and capillary action into the capillary deposition channel 308, which then fills by capillary action between the channel defining surface 304 and the sample pad 30.

Abutment of the sill 302 and the operative surface of the sample pad 30 defines the depth of the capillary deposition channel 308, such that at least a portion of the liquid sample flow from the injection groove portion 290 disperses across the capillary deposition channel 308. Preferably, the capillary deposition channel has a depth equal to or less than 1.0 mm. More preferably, the depth is equal to or less than 0.5 mm, and most preferably the depth is 0.1 mm. The flow of liquid sample is confined to the capillary deposition channel 308 due to the effect of capillary forces, such as a capillary trap, which delay the liquid sample from advancing downstream towards the detection channel 32 beyond the trailing edge 416 of the deposition channel defining surface 304, at least until the deposition channel 308 is substantially filled with sample.

The width M to length N ratio of the deposition channel defining surface 304 is preferably 10. More preferred, the ratio M/N is equal to or less than 7. As such, the surface 304 ends at an air junction edge with the trailing edge 306, that prevents the liquid sample from flowing downstream, thereby promoting the lateral dispersal of the liquid sample by capillary action in the capillary deposition channel308. The liquid sample continues to disperse in and fill the capillary deposition channel 308to form a generally linear sample band. The sample band corresponds to the shape of the deposition channel defining surface 306.

This embodiment of the deposition channel avoids the need to machine or form a capillary channel that is integral within the top member itself, so that sample is distributed uniformly within the channel before being permitted passage onto the carrier. Rather, it is now realized that it is simpler to form a capillary channel, to promote lateral flow of the sample, between the deposition channel defining surface 308 and the sample pad 30. The capillary action causing lateral distribution of the sample is rapid enough that the sample starts to be absorbed into the sample pad 30 across the entire width of the pad 30 almost simultaneously, i.e. the portion of the sample first contacting the pad 30, adjacent the injection channel 204, 304 or injection groove portion 290 does not have any significant lead in absorbing into the pad 30.

In operation, at least a portion of the liquid sample enters the sample deposition means and rapidly disperses by capillary action to the ends of the capillary deposition channel 308. Once the deposition channel 308 has been substantially filled and, accordingly, the capillary draw of the sample pad 30 exceeds that of the deposition channel 308, at least a portion of the sample band advances downstream in the pad 30 towards detection channel 32. The advancing sample band initially advances from deposition channel 308 as a generally linear band which may have a slightly curved liquid frontier with the leading edge at or near the center.

The purpose of the sample deposition means is now apparent. If, in the absence of the capillary trap formed by the deposition channel 308, as defined by the sample pad 30 and the sample deposition channel defining surface 304, an operative surface 312 of sample pad 30 was in direct contact with the injection channel 288, at least a portion of liquid sample in sample delivery means could flow immediately downstream towards detection channel 32. This uncontrolled liquid sample flow would result in a greater amount of red blood cells advancing to the capture zone 38, since a narrow width of the flow of liquid sample would not allow the efficient separation of particulates from the plasma front by operative surfaces of the carriers. Additionally, a liquid sample flow having a narrow width may increase the duration of the diagnostic test, and reduce the concentration of analyte crossing the test line.

Returning to FIG. 21, shown is the configuration of the interior surface 262 of the base member 214. A flat substantially rectangular area 240 and an elongated area 242 projecting from the interior surface 262 of base member 214 hold the porous carrier 24. The height of the sides 244 of the rectangular area 240 is sufficient such that the operative surface 312 of the porous carrier 24 is at the same level as sill 302 of the deposition channel 308. Other configurations are contemplated by the present invention, such as designing the base and top members to hold carrier 24 in the correct orientation such that the recessed rectangular area 240 and elongated area 242 are not necessary. It is only necessary that the base and top members correspond with carrier 24 in between to define the fluid path from the injection channel to detection channel 32. The thickness of the various channels and areas may be adjusted accordingly.

An important advantage of the present invention is the geometry of the device which, when utilized for a blood sample, provides a generally linear sample band on the sample pad 30. The provision of a generally linear sample band. encourages converged flow of the blood sample downstream towards the detection channel 32. A converged flow is beneficial because it forms a flow stream having a red blood cell front and downstream thereof a plasma front.

More specifically, the red blood cells in the blood sample are separated chromatographically from the plasma which, in a carrier 24 composed of, for example, a nitrocellulose membrane, flows faster than the red blood cells. Accordingly, as a result of the geometric design of the device, a relatively larger amount of plasma containing the analyte-binding mobile reagents is separated from the whole blood on sample pad 30, providing sufficient time for binding reactions to occur. When the sample band advances downstream towards the detection pad 32 along the sample pad 30, the pattern of sample band flow tends to converge toward detection pad 32. Additionally, by encouraging a converged flow pattern along sample pad 30, the device concentrates the reagents and the analyte and retards red blood cell migration, and thereby enhances red blood cell/plasma separation, and the sensitivity of the diagnostic test for a given volume of sample.

In use, a user holds the present device in one hand with the top of the device facing the user or tilted somewhat toward the user, i.e. more or less inverted. With the other hand, a drop or more of liquid sample is touched to the inlet and the device is then held in this position until sufficient liquid sample is drawn into the device. For most diagnostic applications, 50 μL of a liquid sample is sufficient to obtain reliable test results. The user is allowed to see the liquid sample advance into and accumulate in injection channel. The device is then returned to an upright position and laid flat on a work surface, and the results of the diagnostic test can then either be viewed or determined instrumentally within about 5-20 minutes by detecting the presence of label at the capture zone 38 corresponding to observation window 22, 122, 222.

It will be appreciated that a device having the present sample delivery features can be operated using a carrier that has features different from those herein described. For instance, and for simplicity, a device having the present sample delivery system can be operated using a carrier that is a single sheet of material. The single sheet of material can be simply rectangular in shape, and accommodated within a housing adapted to receive it. Alternatively, the single sheet of carrier material can be shaped as herein described, to provide a sample pad that is wider than the integral detection pad. This alternative carrier design can readily be accommodated by the housing described herein. It is a preferred embodiment of the present invention that the carrier consist of the array of pads herein described.

It is understood that the present device can be utilized to detect a wide variety of analytes present in numerous different sample types. These include environmental samples such as wastewater, and medical samples that include blood, its components, urine, cerebrospinal fluid, etc. In an embodiment of the present invention, the device is utilized to detect analytes present in whole blood. Such analytes include myoglobin, troponins including TnI, TnT, and TnC, myosin light chain, fatty acid binding protein, actin, CK-MB, CA-III, BNP, and the like, as well as markers of viral, bacterial, fungal and tumour burden, such as PSA, her-1 and her-2. In another embodiment, the device is utilized to detect urine-borne analytes, including hCG, LH, GnRH, drugs of use and abuse, markers of metabolism such as glucose, and the like. The reagents required to conduct assays for these analytes are all available commercially.

In addition, it will be appreciated that the present device can be adapted to detect more than one analyte in a single test. For this purpose, the carrier of the device will comprise mobile, labeled detector reagents for each analyte deposited on the sample pad, and immobilized capture reagents for the resulting analyte complexes, positioned as separate bands or other indicia on the detector pad in full view from window 22, 122, 222.

Use of a device of the present invention is now described in detail in the following examples.

### EXAMPLES

The following results have been obtained by applying the illustrated device in a model system in which the carrier is comprised of the illustrated pad array, where the sample pad is wider than the detection zone formed by the detection pad and bridging pad (absent a wicking pad and without adhesive tape over the detection channel). In the model system, gold conjugated mouse antibody to CK-MB, a cardiac analyte, is used as the labeled detector reagent, which reveals the pattern of sample flow along the carrier. Immobilized goat antibody to CK-MM was used as capture.

More particularly, gold conjugated mouse anti-CKMB (Spectral Diagnostics, Toronto, Canada) solution (OD₅₃₀=40) was prepared by mixing one volume of StabilGuard (SurModics, Inc., Eden Prairie, Minn., USA) and one volume of mouse anti-CKMB gold conjugate (OD₅₃₀=80).

As sample pad, a polyester supported cellulose nitrate membrane (PuraBind, 3 μm nominal pore size; Whatman International Ltd., Maidstone, Kent, UK) was first blocked by immersion into a blocking solution (StabilCoat (SurModics, CA, USA)/H₂O=1/3, v/v). After drying, gold conjugated mouse anti-CKMB antibody (OD₅₃₀=40) was deposited as 0.5 μl dots onto the blocked sample pad by manual pipetting and dried at 37° C. For the detection zone, a polyester supported cellulose nitrate membrane (PuraBind, 5 μm nominal pore size; Whatman. International Ltd., Maidstone, Kent, UK) was first blocked by immersion into a blocking solution (StabilCoat (SurModics, CA, USA)/H₂O=1/3, v/v). After drying, capture line was streaked onto the detection pad using an IsoFlow™ Dispenser (Imagene Technology, Hanover, N.H., USA) with an antibody solution containing 2 mg/ml goat anti-CKMM (Spectral Diagnostics, Inc., Toronto, Canada), 1% sucrose, and 3% methanol.

The carrier was assembled by putting the sample pad and the detection pad with attached bridging pad in the restricted compartments in the base member of the device respectively so that the cellulose nitrate layers of the sample pad and the bridge are facing each other, and the liquid communication between these layers was secured by pressing the upper member of the device housing into the base member.

### Example 1

The converging pattern of sample flow toward the detection channel was first confirmed, in an experiment in which normal human serum was delivered as a linear band from the deposition channel and then permitted to flow for about ten minutes toward the detection channel. The bridge was then removed, thereby stopping sample flow within the sample pad, to reveal the label flow pattern. As illustrated in FIG. 27a, the labeled detector antibodies entrained within the migrating sample clearly displayed flow converging toward the bridging pad of the detection zone. This same converging sample flow is seen in assays that run to completion without bridging pad interruption. Notwithstanding the reduced width of the detection channel relative to the span of labeled detector antibody deposited on the sample pad, there was very little detectable stagnation of reagent or sample in the shoulders of the sample pad. Substantially all of the sample and reagent migrated toward the bridge leading into the detection channel. There is accordingly no need to shape sample deposition channels for confocal sample flow; a linear sample deposition band is sufficient to drive the desired flow into the detection channel.

### Example 2

The carrier pad arrangement just described was also employed in the present device for the detection of rCKMB, as analyte. In this assay, 50 μl of normal human serum alone or spiked with rCKMB was tested. The results at 15 min are illustrated in FIGS. 27b (normal human serum), 27c (normal human serum spiked with 6.25 ng/ml rCKMB), and 27d (spiked with 32.35 ng/ml rCKMB). These Figures reveal that label deposited on the sample pad flowed almost completely out of the sample pad and into the detection zone, there being very marginal and negligible stagnation of labeled reagents in the sample pad shoulders. The results also reveal that rCKMB is detected in the spiked samples, the assay being more sensitive to detection of CKMB at the higher concentrations.

### Example 3

The device also was assessed for its ability to retard the flow of red blood cells, so that they do not migrate into and obscured results otherwise visible at the capture line. To this end, 50 μl of fresh heparinized human whole blood was tested. After 15 min, the majority of the red blood cells were retained in the sample pad, and the front of the red blood cells was restricted at the center of the bridging pad. After about 1 hour, the front of the red blood cells was stabilized at just beyond the bridge before reaching the capture line within the read-out window, and remained there afterwards. Hemolysis was not visually detectable. The result at 72 hours is shown in FIG. 27e. It will thus be appreciated that the bridging pad and its elevation relative to the sample and detection pads also contributes to the filtration of sample particulates including red blood cells, and that this carrier pad array is particularly well adapted for detection of soluble analytes present in blood samples.

While the invention has been exemplified with reference to a particular diagnostic assay and format, it will be appreciated that any of a variety of lateral flow type assays can be conducted to detect a variety of analytes present in different liquid samples.

1. A device of the type useful to perform a lateral flow, immunodiagnostic assay, the device comprising;
a carrier for conducting flow of liquid sample suspected of containing an analyte of interest
one or more reagents disposed on said carrier, for detecting said analyte; and
sample deposition means associated with said carrier, said sample deposition means being adapted to deposit sample onto said carrier as a sample band that is essentially linear and is generally transverse to the desired direction of subsequent sample flow.

2. A device according to claim 1, further comprising a detection channel defined downstream of said sample band, the sample band having a width greater than the detection channel, said channel further comprising an immobilized reagent for binding with said analyte or with a binding partner thereof.

3. A device according to claim 1, wherein said carrier comprises a sample receiving pad, an analyte detection pad, and a bridging pad connecting the sample receiving pad and analyte detection pad in flow communication.

4. A device according to claim 3, further comprising a wettable barrier layer adhered to and connecting the bridging pad and the analyte detection pad.

5. A device according to claim 3, wherein the bridging pad is positioned in contact with and above the sample pad and detection pad.

6. A device of the type useful to perform a lateral flow immunodiagnostic assay for one or more target analytes, the device comprising:
a carrier for conducting the flow of sample along a liquid path; the carrier comprising a sample receiving zone comprising a detectably labeled, mobile detector reagent for binding to the target analyte, and an analyte detection zone downstream of the sample receiving zone and comprising an immobilized capture reagent for binding with a complex formed between said analyte and said capture reagent, and
a housing for receiving the carrier, the housing comprising a sample receiving means and a sample deposition means in flow communication with the sample receiving means and the sample receiving zone, the sample deposition means being adapted to deposit liquid sample as a sample band that is essentially linear and has a width greater than the width of the analyte detection zone.

7. A device according to claim 6, wherein the sample receiving zone and the analyte detection zone are defined on distinct carrier pads, and wherein said pads are coupled for sample flow by a bridging pad.

8. A device according to claim 7, wherein the bridging pad and the analyte detection zone pad are connected by a wettable barrier layer that enhances capillary flow therealong.

9. A device according to claim 8, wherein the bridging pad has a width narrower than the length of the sample band deposited from the sample deposition means.

10. A device according to claim 8, wherein the sample deposition means comprises a sample inlet means and a sample deposition channel, for moving sample from the inlet to the sample receiving zone.

11. A device according to claim 10, wherein the sample deposition channel is integrated within the housing.

12. A device according to claim 10, wherein the sample deposition channel is dimensioned to promote lateral capillary flow to fill the sample deposition channel, before any substantial flow of the sample into the carrier.

13. A device according to claim 12, wherein the sample deposition channel is formed substantially within the housing.

14. A device according to claim 12, wherein the sample deposition channel is defined between a deposition channel defining surface and the carrier.

15. A diagnostic device of the type useful to perform a lateral flow immunodiagnostic assay, the device comprising a carrier for conducting the flow of sample therealong, the carrier comprising a sample pad for receiving the sample and, in flow communication therewith, a detection pad comprising at least one reagent useful in detecting analyte in the sample, wherein the width of the sample pad is greater than the width of the detector pad.

16. A diagnostic device according to claim 15, wherein the carrier further comprises a bridging pad, and further wherein the bridging pad couples the sample pad and the detector pad in flow communication.

17. A diagnostic device according to claim 16, wherein each pad in said carrier comprises a water-impermeable backing layer and a sample conducting face, and further wherein the sample conducting face of the bridging pad is in contact with the sample conducting faces of each of the detection pad and the sample pad.

18. A diagnostic device according to claim 16, wherein the bridging pad and the detection pad are coupled in flow communication by an upper barrier layer of water-impermeable material that is either translucent or transparent.

19. A diagnostic device according to of claim 15, further comprising a housing adapted to deposit sample onto said sample pad as a generally linear band having an axis that is transverse to the direction of sample flow and is wider than the width of the detection pad.

20. A diagnostic device for testing a liquid sample, the device comprising:
(a) a carrier for receiving at least a portion of the sample; and
(b) a sample delivery means, said sample delivery means having a delivery channel that is in fluid communication with the carrier, said delivery channel having a first delivery channel surface facing a second delivery channel surface, wherein said first delivery channel surface is spaced apart from said second delivery channel surface by a distance that promotes longitudinal advancement of the sample along the delivery channel by capillary action.

21. The device of claim 20, wherein the distance between the first delivery channel surface and the second delivery channel surface is less than 0.5 mm.

22. The device of claim 21, wherein the distance between the first delivery channel surface and the second delivery channel surface is less than 1.0 mm.

23. The device of claim 20, wherein the delivery channel has an inlet and an outlet, wherein at least a portion of the sample received at the inlet advances between the first delivery channel surface and the second delivery channel surfaces toward the outlet.

24. The device of claim 20, wherein the delivery channel is in fluid communication with a sample deposition means, wherein at least a portion of the sample received at the outlet of the delivery channel flows to the sample deposition means.

25. The device of claim 24, wherein the sample deposition means includes a deposition channel, said deposition channel having a deposition channel defining surface facing the carrier and defining the deposition channel, and wherein the deposition channel has a depth that promotes lateral distribution of the sample along the deposition channel by capillary action, thereby to form a sample band across the carrier.

26. The device of claim 25, wherein the depth of the deposition channel between the deposition channel defining surface and the carrier is less than 0.5 mm.

27. The device of claim 26, wherein the depth of the deposition channel between the deposition channel defining surface and the carrier is less than 1.0 mm.

28. The device of any one of claims 25, wherein the first delivery channel surface is formed with an advancement groove, wherein at least a portion of the sample received at the inlet advances within the advancement groove along the delivery channel to the outlet.

29. The device of claim 28, wherein the advancement groove extends beyond the outlet of the delivery channel to promote the flow of at least a portion of the sample into the sample deposition channel.

30. The device of claims 24, wherein the delivery channel is generally rectilinear.

31. The device of claims 24, wherein the volumetric capacity of the sample delivery means provides a sufficient amount of liquid sample for the testing.

32. The device of any one of claims 24 to 31, including a housing enclosing the carrier, wherein the sample delivery means and the sample deposition means are formed in the housing.

33. The device of claim 32, wherein the housing comprises a base member and an upper member, wherein the first delivery channel surface is provided on the base member and the second delivery channel surface is provided on the upper member.

34. The device of claim 33, wherein the base and upper members include abutting surfaces determining the distance between the first and second delivery channels.

35. The device as claimed in claims 20, wherein the carrier includes at least one detachably labeled, mobile reagent for binding with a target analyte and, at a position downstream thereof, at least one immobilized capture reagent for binding with the target analyte or a complex formed therewith.

36. A diagnostic device for testing a liquid sample, the device comprising:
(a) a carrier for receiving at least a portion of the sample; and
(b) a sample injection means, said sample injection means having an injection channel that is in fluid communication with the carrier.

37. The device of claim 38, wherein the injection channel is in fluid communication with a sample delivery means, and wherein at least a portion of the sample received in the sample delivery means flows into the injection channel.

38. The device of claim 37, wherein the sample delivery means includes an advancement groove, wherein the advancement groove extends into the injection channel to promote the flow of at least a portion of the sample into the injection channel.

39. The device of claim 38, wherein the volumetric capacity of the injection channel provides a sufficient amount of liquid sample for the testing.

40. The device of claim 36, wherein the carrier includes at least one detectably labeled, mobile detection reagent for binding to a target analyte, and, downstream therefrom, at least one immobilized capture reagent for binding with a complex formed by a corresponding mobile detection reagent and said target analyte.
